SEQUENCE LISTING (1 sequence)

SEQ ID NO: 1 - protein, 45 aa, homo sapiens
AERIVRGQTAESLAVHNITVCEQKISEWGKIKECPIPQFAGNPPM

(19) United States
(12) Patent Application Publication
(10) Pub. No.: US 2021/0169910 A1
(43) Pub. Date: Jun. 10, 2021

(54) OLEUROPEIN FOR USE IN REDUCING POST-PRANDIAL GLYCAEMIA

(71) Applicant: Università degli Studi di Roma "La Sapienza", Roma (IT)

(72) Inventors: Francesco Violi, Roma (IT); Roberto Carnevale, Fondi (LT) (IT); Cristina Nocella, Formia (LT) (IT); Daniele Pastori, Roma (IT)

(21) Appl. No.: 16/770,610

(22) PCT Filed: Dec. 4, 2018
(86) PCT No.: PCT/IB2018/059608

§ 371 Date: Jun. 7, 2020

(30) Foreign Application Priority Data
Dec. 7, 2017 (IT) 102017000141832

(51) Int. Cl. A61K 31/7048; A61P 39/06; A61P 3/10

(52) U.S. Cl. CPC A61K 31/7048; A61P 3/10; A61P 39/06

(57) ABSTRACT

Oleuropein for use in reducing post-prandial glycaemia in healthy subjects.

### FIELD OF THE INVENTION

The disclosure concerns use of oleuropein for reducing post-prandial glycaemia.

### BACKGROUND OF THE INVENTION

Observational as well as intervention studies demonstrated that Mediterranean diet is associated with a lower risk of cardiovascular events (Salas-Salvadó et al., 2014). Mediterranean diet is rich in fruits and vegetables, has low content of meat and dairy and includes extra virgin olive oil (EVOO) and small amount of wine (Gerber et al., 2015). Recent studies demonstrated that, among the typical nutrients of Mediterranean diet, EVOO possess beneficial effects as documented by reduction of cardiovascular events in patients at risk of atherosclerosis given a Mediterranean diet supplemented with EVOO (Salas-Salvadó et al., 2014). Prevention of new onset diabetes has been suggested as a mechanism potential retarding atherosclerotic progression and its clinical sequelae by EVOO (Babio et al., 2014).

Olive oil is composed mainly of the mixed triglyceride esters of oleic acid and palmitic acid and of other fatty acids, along with traces of squalene (up to 0.7%) and sterols (about 0.2% phytosterol and tocosterols). Olive oil also contains phenolic compounds, such as esters of tyrosol, hydroxytyrosol, oleocanthal and oleuropein, having acidic properties that give extra-virgin unprocessed olive oil its aroma and bitter, pungent taste. Olive oil is a source of at least 30 phenolic compounds, among which is elenolic acid. Oleuropein, together with other closely related compounds such as 10-hydroxyoleuropein, ligstroside and 10-hydroxyligstroside, are tyrosol esters of elenolic acid. Other phenolic constituents include flavonoids, lignans and pinoresinol.

In view of the beneficial effects exerted by EVOO, there is the need to identify the most important component(s) of EVOO which is(are) able to reduce adverse cardiovascular events through control of post-prandial glucose and oxidative stress, in order to allow a more easy assumption of these components also in areas different from the Mediterranean one, wherein EVOO is not easily available.

### SUMMARY OF THE INVENTION

The object of this disclosure is to provide a novel aid in controlling post-prandial glycaemia.

According to the invention, the above object is achieved thanks to the subject matter recalled specifically in the ensuing claims, which are understood as forming an integral part of this disclosure.

The present invention provides a novel use of oleuropein in reducing post-prandial glycaemia, preferably in healthy subjects.

### BRIEF DESCRIPTION OF THE DRAWINGS

The invention will now be described in detail, purely by way of illustrative and non-limiting example, with reference to the attached figures, wherein:

FIG. 1. Simplified flow chart of the cross-over study design.

FIG. 2. Effect of oleuropein on oxidative stress biomarkers. sNox2dp (n=20) (A), 8-iso-PGF2a (n=20) (B), platelet p47^{phox} phosphorylation (n=5) densitometry with a representative western blot bands (C) before (T0) and 2 h after meal (T120) in healthy subjects supplemented with 20 mg oleuropein (black dotted line) or 20 mg placebo (black line) (*p<0.001; **p<0.05).

FIG. 3. Effect of oleuropein on glycaemic profile.

Blood glucose (A), insulin (B), GLP1 (C), DPP-4 activity (D) before (TO) and 2 h after meal (T120) in healthy subjects (n=20) supplemented with 20 mg oleuropein (black dotted line) or 20 mg placebo (black line) (*p<0.001; **p<0.05)

### DETAILED DESCRIPTION OF THE INVENTION

In the following description, numerous specific details are given to provide a thorough understanding of embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment.

Furthermore, the particular features, structures, or characteristics may be combined in any suitable manner in one or more embodiments.

The headings provided herein are for convenience only and do not interpret the scope or meaning of the embodiments.

In one embodiment, the instant disclosure concerns use of oleuropein for reducing post-prandial glycaemia, preferably in healthy subjects.

In one or more embodiments, oleuropein is for administration before a meal, preferably immediately before a meal.

According to further embodiments, oleuropein is for administration at a daily dose of 10 to 40 mg, preferably 20 to 30 mg.

In one or more embodiments, the administration of oleuropein counteracts formation of reactive oxidant species through down regulation of Nox2 activation during the post-prandial phase.

In one or more embodiments, the administration of oleuropein up regulates insulin secretion and reduces blood glucose during the post-prandial phase.

The present description provides the first experimental evidence that oleuropein, a component of EVOO, is an antioxidant molecule, which lowers post-prandial glycaemia with an oxidative stress-mediated mechanism involving Nox2 down-regulation.

There is a growing body of evidence to suggest that post-prandial phase is associated with an abrupt formation of reactive oxidant species (ROS), which are implicated in systemic inflammation, endothelial dysfunction and eventually cardiovascular disease (b Carnevale et al., 2014, Lahera et al., 2007). Analysis of Nox2 activity, which is among the most important cellular producer of ROS (Violi et al., 2017), allowed the present inventors to demonstrate that post-prandial formation of ROS was attributable to Nox2 activation and that EVOO was able to counteract this phenomenon by down-regulating Nox2 activation; this effect seemed to be devoted to specific but unknown components of EVOO, as corn oil was unable to protect against post-prandial oxidative stress (b Carnevale et al., 2014).

The present disclosure shows for the first time that administration of 20 mg oleuropein to healthy subjects is associated with an antioxidant effect.

Several methodologies were employed by the present inventors to explore the antioxidant activity of oleuropein. In particular, it has been shown that 20 mg of oleuropein down-regulated Nox2 activation, as demonstrated by the reduction in sNox2-dp and platelet p47^{phox} phosphorylation, the cytosolic sub-unit of Nox2 that activates the catalytic site after translocation to the cell membrane. Moreover, the reduction of oxidative stress was corroborated by decreased production of 8-iso-PGF2α.

ROS formation during the post-prandial phase may have potential deleterious effect on glycaemic metabolism as ROS can interfere with incretin production by gastro-intestinal tube. Thus, incretins, like GLP1 and GIP, are secreted by distal small intestine in response to its stimulation and bind receptors in the endocrine pancreas so eliciting insulin secretion and lowering post-prandial blood glucose (Seino et al., 2010). ROS are important regulator of this phenomenon by modulating the activity of DPP-4, which rapidly inactivates incretin activity so impairing insulin secretion (Rhee et al., 2014; Smilowitz et al., 2014).

The present disclosure provides for the first time evidence that supplementation of a meal with EVOO had positive effects on post-prandial glycaemic profile as it was associated with an increase of incretins coincidentally with a decrease of DPP-4 activity. This finding leads to suggest that EVOO behaves as a DPP-4 inhibitor and that EVOO was responsible for improved post-prandial glycaemia via an oxidative stress-mediated mechanism and eventually incretin up-regulation (Violi et al., 2015).

The present disclosure investigated if this phenomenon could be attributed to a EVOO component. To address this issue, the effect of 20 mg oleuropein versus placebo on post-prandial glycaemic profile of healthy subjects was investigated.

The present disclosure demonstrates that oleuropein treatment was associated with an average 14 mg/dL reduction of glucose. As this effect was coincident with a significant increase of insulin, it has been considered that oleuropein could be the EVOO component responsible for incretin up-regulation. In accordance with this, blood activity of GLP1 was significantly increased after oleuropein treatment while DPP4 activity significantly decreases suggesting that oleuropein behaves as a DDP4 inhibitor via lowering Nox2-derived oxidative stress.

The instant disclosure provides the first evidence that EVOO encompasses a component that improves the post-prandial glycaemic control in healthy subjects via an oxidative stress-mediated mechanism.

Results

Post-Prandial Oxidative Stress

Clinical characteristics of the population are reported in Table 1.

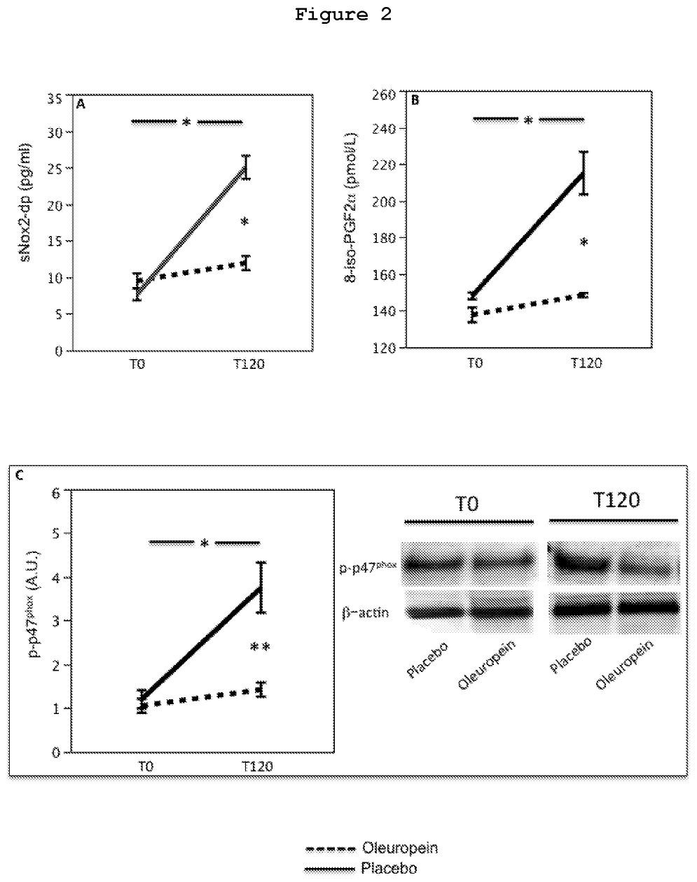

Baseline blood variables were similar in the two groups (FIG. 2 Panels A-C). A significant difference between meal with oleuropein or placebo was found with respect to sNox2-dp (*p<0.001; FIG. 2A), 8-iso-PGF2a (*p<0.001; FIG. 2B) and platelet p47^{phox} phosphorylation (**p<0.05; FIG. 2C).

Compared to baseline, placebo-treated subjects showed increased sNox2-dp (Δ change 226%), 8-iso-PGF2a (Δ change 45%) and platelet p47^{phox} phosphorylation (A change 212%) (FIG. 2A-C). Conversely, compared to baseline, oleuropein-treated subjects showed a less increase of sNox2-dp (Δ change 25%), 8-iso-PGF2α (Δ change 8%) and platelet p47^{phox} phosphorylation (Δ change 42%) (FIG. 2A-C).

Post-Prandial Glycaemic Profile

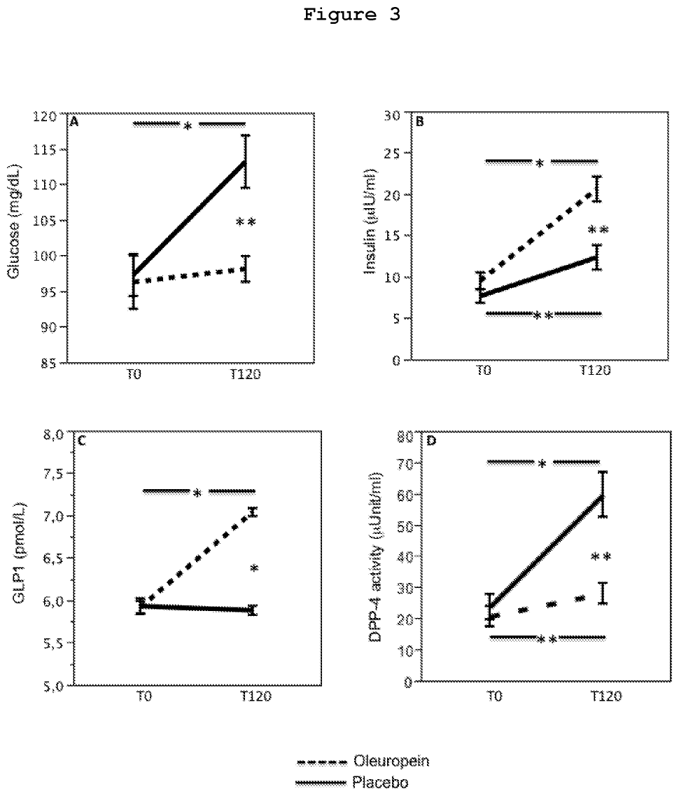

Baseline blood variables were similar in the two groups (FIG. 3 Panels A-D). A significant difference between meal with oleuropein or placebo was found with respect to glucose (**p<0.05; FIG. 3A), insulin (**p<0.05; FIG. 3B), GLP1 (*p<0.001; FIG. 3C) and DPP-4 activity (**p<0.05; FIG. 3D).

Compared to baseline, placebo-treated subjects showed increased levels of glucose (Δ change 16%) and insulin (Δ change 61%) (FIG. 3 A-B); conversely, compared to baseline, in subjects given oleuropein, a less increase of blood glucose (Δ change 2%) and a more marked increase of blood insulin (Δ change 116%) was observed (FIG. 3A-B). Compared to baseline, GLP1 increased significantly in oleuropein-treated subjects (Δ change 19%) while it unchanged after placebo (Δ change −1%). Furthermore, DPP-4 activity showed a marked increase in placebo-treated subjects (Δ change 150%) and a less increase in oleuropein-treated ones (Δ change 35%) (FIG. 3 C-D). Δ of GLP1 correlated with Δ of DPP-4 activity (Rs:-0.558; p<0.001) and Δ insulin (Rs: 0.330; p<0.05). Δ of DPP-4 activity correlated with Δ sNox2dp (Spearman's rho (Rs): 0.615; p<0.001), Δ 8-iso-PGF2α (Spearman's rho (Rs): 0.33; p<0.05) and Δ platelet p47^{phox} phosphorylation (Spearman's rho (Rs): 0.435; p<0.05).

Materials and Methods

Study Population and Recruitment

Twenty healthy subjects (HS) (10 males and 10 females, age 33.9±6.9) gave informed consent to participate in the interventional study, which was performed between July-September 2016 at Policlinico Umberto Rome. Clinical and demographics characteristics of HS as well as nutrient lunch are reported in Table 1 and Table 2, respectively.

TABLE 1
Baseline characteristics
of Healthy Subjects

| | Healty |
|---|---|
| | Subjects |
| Variables | (n = 20) |
| Age (years) | 33.9 ± 6.9 |
| Males n (%) | 10 (50) |
| BMI (kg/m²) | 20.7 ± 3.7 |
| Systolic BP (mmHg) | 124 ± 5.0 |
| Diastolic BP (mmHg) | 72 ± 7.4 |
| Total Cholesterol | 171 ± 13.3 |
| (mg/dl) | |
| LDL (mg/dl) | 81 ± 11.3 |
| Fasting glycaemia | 91 ± 7.1 |
| (mg/dl) | |

TABLE 2
Bromatological composition of the meal

| | Gr | Proteins | Lipids | Carbohydrates |
|---|---|---|---|---|
| Pasta | 100 | 10.8 | 0.3 | 82.8 |
| Chicken | 150 | 35.4 | 2.1 | 0 |
| breast | | | | |
| Salad | 80 | 1.44 | 0.32 | 1.76 |
| Bread | 80 | 6.56 | 0.4 | 54 |
| Apple | 200 | 0.4 | 0.6 | 22 |
| Total Gr. | | 54.6 | 3.72 | 160.56 |
| Calories | | 218.4 | 33.48 | 642.24 |

Study Design

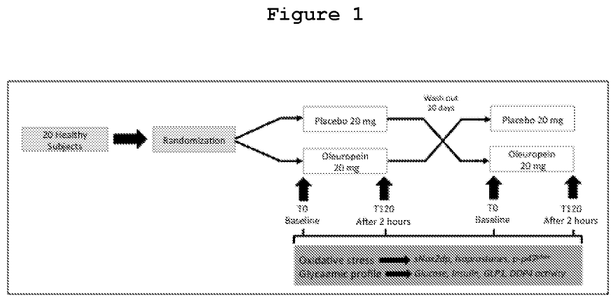

The design was a randomized, double blind, placebo controlled, crossover study (FIG. 1). Participants were randomized to receive 20 mg of oleuropein or 20 mg of placebo immediately before a lunch. The randomization was carried out by a procedure based on a random numeric sequence. Placebo and active capsules were identical in appearance and both were odourless. The quality control on both capsules was performed confirming their safety. None of the participants were receiving any vitamin or antioxidant supplements, statin or antiplatelet drugs in the month preceding the beginning of the study. Others exclusion criteria were: i) presence of malignancy, ii) presence of chronic inflammatory diseases, iii) alcohol intake, iv) smoking, v) pregnant or breast-feeding, vi) age less than 18 years. After a 10 days washout phase, participants crossed over to take the opposite intervention. Participants were assessed at baseline (T0) and two hours after lunch (T120). Glycaemic profile, which included glucose, insulin, dipeptidyl-peptidase-4 (DPP-4) activity and glucagon-like peptide-(GLP-1) and oxidative stress, which included sNox2-dp, 8-iso-PGF2α and platelet p47phox phosphorylation, were analysed before and two hours after meal. Every blood determination was performed blind.

The study was conformed to the declaration of Helsinki and approved by the Ethical Committee of Sapienza University of Rome (n° 509/16).

Serum Nox2

Serum Nox2 was measured as soluble Nox2-derived peptide (sNOX2dp) with an ELISA method, which was partly modified in comparison to that previously reported (Pignatelli et al., 2010; WO 2010142794 A1).

The assay is based on:

1) coating reference standards of known concentrations of sNox2dp and of serum samples (1 μg of protein) into ELISA 96 well plate overnight at 4° C.,

2) wash away of unbound materials from samples,

3) addition in each well of anti-sNox2dp-horseradish peroxidase (HRP) monoclonal antibody against an epitope present on the extra membrane portion of Nox2 and specifically against the portion of Nox2 protein having the amino acid sequence set forth in SEQ ID No.:1 (deposited on 17 Oct. 2017 by CBA-ICLC of Genova, Italy, under deposit number PD17002), and

4) quantification of immobilized antibody enzyme conjugates by monitoring HRP activities in the presence of the substrate 3,3′,5,5′-tetramethylbenzidine (TMB). The enzyme activity is measured spectrophotometrically by the increased absorbency at 450 nm after acidification of formed products (2 M sulphuric acid). Since the increase in absorbency is directly proportional to the amount of sNox2dp of the unknown sample, the latter can be derived by interpolation from a reference curve generated in the same assay with reference standards of known concentrations of sNox2dp (0-200 pg/ml). Values were expressed as pg/ml; intra-assay and inter-assay coefficients of variation were <10%.

8-Iso-PGF2α Assays

Serum isoprostanes (8-iso-PGF2α-III) were measured by the enzyme immunoassay method (DRG International) and expressed as pmol/L. Intra-assay and inter-assay coefficients of variation were <10%.

Western Blot Analysis of p47^{phox} Phosphorylation

p47^{phox} phosphorylation was analysed in platelets prepared as previously described [13 Carnevale et al., 2014 a)]. Platelet pellets were suspended in a 2× Lysis buffer (5 mM EDTA, 0.15 mol NaCl, 0.1 mol Tris pH 8.0, 1% triton and 10 μg/ml of protease and phosphatase inhibitors cocktail). Equal amounts of protein (30 μg/lane) estimated by Bradford protein assay were solubilized in a 2× Leammli sample buffer containing 20% of 2-mercaptoethanol. Proteins were separated by SDS-PAGE on 10% polyacrylamide gel and then electro-transferred to nitrocellulose membranes. After blocking, membranes were incubated with rabbit polyclonal anti-p47^{phox} antibody to phosphoserine (Abcam, Product Code ab74095) or mouse monoclonal anti-βactin antibody (Santa Cruz Biotechnology—Product Code sc-47778) and incubated overnight at 4° C. Then, the membranes were incubated with goat anti-rabbit secondary antibody (Santa Cruz Biotechnology—Product Code sc2004, 1:5000) or with goat anti-mouse secondary antibody (Santa Cruz Biotechnology—Product Code sc2005, 1:5000) and then the immune complexes were detected by enhanced chemiluminescence substrate. Densitometric analysis of the bands was performed using Image J software.

Serum Glucose and Insulin Concentration

Glucose and insulin were measured in serum samples using ELISA commercial Kit (Arbor Assay and DRG International, respectively). Glucose values were expressed as mg/dl whereas insulin values were expressed as μIU/ml.

Serum Glucagon Like Peptide-1 (GLP1) Concentration

Commercial ELISA Kit (DRG International) was used for the quantitative determination of bioactive GLP1 (7-36) and (9-36) levels in serum. GLP1 values are expressed as pmol/L.

Dipeptidyl Peptidase-4 (DDP4) Activity

DDP4 activity was evaluated in serum samples by a commercial assay (Sigma Aldrich). In this assay, DPP4 cleaves a non-fluorescent substrate, H-Gly-Pro-AMC, to release a fluorescent product, 7-Amino-4-Methyl Coumarin (AMC). DDP4 activity was expressed as μU/ml, where one unit of DPP4 is the amount of enzyme that will hydrolyze the DPP4 substrate to yield 1.0 μmole of AMC per minute at 37° C.

Sample Size

The minimum number of participants that will be included in this cross-over study was calculated considering: 1) relevant difference of postprandial sNOX2-dp levels between the two groups (treated with oleuropein vs. placebo) (after 120 minutes) ≥12 μg/ml; 2) SDs homogeneous between the groups (=11) and 3) type I error probability α=0.05 and power 1-β=0.90. The minimum sample size for this crossover study was 20 subjects.

Statistical Methods

Categorical variables are reported as counts (percentage) and continuous variables as means±s.d. unless otherwise indicated. Independence of categorical variables was tested by χ2-test. Comparisons between groups were carried out by Student's t-test and were replicated as appropriate with nonparametric tests (Kolmogorov-Smirnov (z) test in case of nonhomogeneous variances as verified by Levene's test). The cross-over study data were analyzed for the assessment of treatment and period effects, by performing a split-plot ANOVA with one between-subject factor (treatment sequence) and two within-subject factors (pre- vs post-treatment). The analysis was performed separately to compare a meal with and without oleuropein. Results were expressed as means±SD. Bivariate analysis was performed by Spearman rank correlation test. A value of p<0.05 was considered statistically significant. All analyses were carried out with SPSS V.18.0 (SPSS Statistics v. 18.0, SPSS Inc., Chicago, Ill., USA).

### REFERENCES

Babio, N., Toledo, E., Estruch, R., Ros, E., Martínez-Gonźalez, M. A., Castaner, O., Bullo, M., Corella, D., Aros, F., Gómez-Gracia, E., Ruiz-Gutiérrez, V., Fiol, M., Lapetra, J., Lamuela-Raventos, R. M., Serra-Majem, L., Pintó, X., Basora, J., Sorlí, J. V., Salas-Salvadó, J., PREDIMED Study Investigators. (2014). Mediterranean diets and metabolic syndrome status in the PREDIMED randomized trial. CMAJ, 186, E649-57.

a Carnevale, R., Bartimoccia, S., Nocella, C., Di Santo, S.,

Loffredo, L., Illuminati, G., Lombardi, E., Boz, V., Del Ben, M., De Marco, L., Pignatelli, P., Violi, F. (2014). LDL oxidation by platelets propagates platelet activation via an oxidative stress-mediated mechanism. Atherosclerosis, 237, 108-16.

b Carnevale, R., Pignatelli, P., Nocella, C., Loffredo, L., Pastori, D., Vicario, T., Petruccioli, A., Bartimoccia, S., Violi F. (2014). Extra virgin olive oil blunt post-prandial oxidative stress via NOX2 down-regulation. Atherosclerosis, 235, 649-58.

Gerber, M., Hoffman, R. (2015). The Mediterranean diet: health, science and society. Br J Nutr, 113, S4-10.

Lahera, V., Goicoechea, M., de Vinuesa, S.G., Miana, M., de las Heras, N., Cachofeiro, V., Luño, J. (2007). Endothelial dysfunction, oxidative stress and inflammation in atherosclerosis: beneficial effects of statins. Curr Med Chem, 14, 243-8.

Pignatelli, P., Carnevale, R., Cangemi, R., Loffredo, L., Sanguigni, V., Stefanutti, C., Basili, S., Violi, F. (2010). Atorvastatin Inhibits gp9lphox Circulating Levels in Patients With Hypercholesterolemia. Arterioscler Thromb Vasc Biol, 30, 360-7.

Rhee, N. A., Ostoft, S. H., Holst, J. J., Deacon, C. F., Vilsboll, T., Knop, F. K. (2014). The impact of dipeptidyl peptidase 4 inhibition on incretin effect, glucose tolerance, and gastrointestinal-mediated glucose disposal in healthy subjects. Eur J Endocrinol, 171, 353-62.

Salas-Salvadó, J., Bulló, M., Estruch, R., Ros, E., Covas, M. I., Ibarrola-Jurado, N., Corella, D., Arós, F., Gómez-Gracia, E., Ruiz-Gutiérrez, V., Romaguera, D., Lapetra, J., Lamuela-Raventos, R. M., Serra-Majem, L., Pintó, X., Basora, J., Munõz, M. A., Sorlí, J. V., Martinez-González, M. A. (2014). Prevention of diabetes with mediterranean diets: a subgroup analysis of a randomized trial. Ann Intern Med, 160, 1-10.

Seino, Y., Fukushima, M., Yabe, D. (2010). GIP and GLP-1, the two incretin hormones: Similarities and differences. J Diabetes Investig, 1, 8-23.

Smilowitz, N. R., Donnino, R., Schwartzbard, A. (2014). Glucagon-like peptide-1 receptor agonists for diabetes mellitus: a role in cardiovascular disease. Circulation, 129, 2305-12.

Violi, F., Carnevale, R., Loffredo, L., Pignatelli, P., Gallin, J. I. (2017). NADPH Oxidase-2 and Atherothrombosis: Insight From Chronic Granulomatous Disease. Arterioscler Thromb Vasc Biol, 37, 218-25.

Violi, F., Pignatelli, P. In vitro method for detecting gp91phox as a marker of oxidative stress. WO 2010142794 A1.

1. A method for reducing post-prandial glycaemia in a subject in need thereof, comprising administering oleuropein to the subject.

2. The method according to claim 1, wherein oleuropein is administered before a meal.

3. The method according to claim 1, wherein oleuropein is administered at a daily dose of 10 to 40 mg.

4. The method according to claim 1, wherein oleuropein is administered to a healthy subject.

5. The method according to claim 1, wherein oleuropein counteracts formation of reactive oxidant species through down regulation of Nox2 activation during the post-prandial phase.

6. The method according to claim 1, wherein oleuropein up regulates insulin secretion and reduces blood glucose during the post-prandial phase.
